(19) United States
(12) Patent Application Publication
(10) Pub. No.: US 2026/0283589 A1
(43) Pub. Date: Sep. 24, 2026

(54) METHOD AND APPARATUS FOR DETECTING MICROEMBOLIC SIGNALS AMONG DOPPLER ULTRASOUND SIGNALS USING ARTIFICIAL INTELLIGENCE

(71) Applicant: SHMD.LTD, Cheongju-si Chungcheongbuk-do (KR)

(72) Inventor: Minyeong SONG, Seoul (KR)

(73) Assignee: SHMD.LTD, Cheongju-si Chungcheongbuk-do (KR)

(21) Appl. No.: 19/681,406

(22) Filed: May 19, 2026

Related U.S. Application Data
(63) Continuation of application No. PCT/KR2025/002851, filed on Feb. 28, 2025.

(30) Foreign Application Priority Data
Jul. 3, 2024 (KR) 10-2024-0087210
Oct. 17, 2024 (KR) 10-2024-0142533
Feb. 28, 2025 (KR) 10-2025-0026815

(51) Int. Cl. A61B 8/08; A61B 8/00

(52) U.S. Cl. CPC A61B 8/085; A61B 8/4477; A61B 8/488; A61B 8/54

(57) ABSTRACT

According to one aspect of the present invention, a method for providing information on whether microemboli exist within a subject using Doppler ultrasound is provided, the method comprising: acquiring, by using a first probe, first Doppler ultrasound measurement results for a measurement target area of the subject; preprocessing, by a processor, the first Doppler ultrasound measurement results to obtain preprocessed first Doppler ultrasound measurement results; determining, by the processor, whether microembolic signals (MES) are present in the preprocessed first Doppler ultrasound measurement results by using a first artificial intelligence algorithm; and providing, by the processor, information on microemboli within the subject according to whether the MES are present.

### CROSS-REFERENCE TO RELATED APPLICATIONS

This application is a continuation of International Application No. PCT/KR2025/002851 filed on Feb. 28, 2025, which claims priority to Korean Patent Application Nos. 10-2024-0087210 filed on Jul. 3, 2024, 10-2024-0142533 filed on Oct. 17, 2024, 10-2025-0026815 filed on Feb. 28, 2025, the entire contents of which are herein incorporated by reference.

### TECHNICAL FIELD

The present invention relates to a method and device for detecting microembolic signals (MES) from Doppler ultrasound signals using artificial intelligence (AI).

### BACKGROUND ART

An embolus is a substance that travels through the bloodstream and blocks a blood vessel. It may take various forms such as a blood clot, fat tissue, air bubbles, or other foreign materials. When an embolus occludes a vessel, it can cause serious diseases such as stroke, pulmonary embolism, and myocardial infarction.

A microembolus is very small and may not cause distinct clinical symptoms at an early stage; however, its continual accumulation or migration may lead to fatal diseases over time. In Doppler ultrasound imaging, microemboli appear as microembolic signals (MES) and can serve as early predictive indicators of embolus formation, vascular occlusion, and cancer metastasis.

Conventional Doppler ultrasound devices are bulky, heavy, and expensive, and their operation requires trained medical personnel, which makes personal use by patients difficult. Thus, there is a need for a Doppler ultrasound device that is portable and easy to use by non-specialists.

### DISCLOSURE INVENTION

### Technical Problem

An object of the present invention is to provide a method and system that detect MES from Doppler ultrasound signals using artificial intelligence.

### Technical Solution

Representative features of the present invention for achieving the above purpose are as follows.

In a representative aspect, a method is provided for offering information as to whether microemboli are present within a subject using Doppler ultrasound, the method comprising: using a first probe on a measurement target area of the subject to acquire first Doppler ultrasound measurement results; by a processor, preprocessing the first Doppler ultrasound measurement results to obtain preprocessed first Doppler ultrasound measurement results; by the processor, determining, using a first artificial intelligence algorithm, whether microembolic signals (MES) are present in the preprocessed first Doppler ultrasound measurement results; and by the processor, providing information about microemboli within the subject based on the determination of whether the MES are present.

In another aspect, an MES detection device is provided that includes a sensor module with a first probe, a storage that stores a first AI algorithm, and a processor configured to: acquire first Doppler ultrasound measurement results using the first probe at the subject's measurement target area; preprocess the first Doppler ultrasound measurement results to obtain preprocessed first Doppler ultrasound measurement; determine, using the first AI algorithm, whether MES are present in the subject from the preprocessed first Doppler ultrasound measurement; and provide information about microemboli within the subject based on the determination of whether the MES are present.

Other methods and devices for implementing the invention and a non-transitory computer-readable recording medium storing a computer program for executing the methods are also provided.

### Advantageous Effects

According to the present invention, the presence of MES can be determined rapidly and accurately by applying AI to signals obtained via Doppler ultrasound, thereby contributing to early diagnosis and prevention of diseases related to microemboli.

According to the present invention, by combining a portable and user-friendly Doppler ultrasound device with AI technology, even non-medical professionals can easily use the system, allowing continuous health monitoring at home.

### BRIEF DESCRIPTION OF THE DRAWINGS

FIG. 1 is a block diagram of an MES detection device according to an embodiment of the invention.

FIG. 2 is a flowchart illustrating an operating method of the MES detection device according to an embodiment of the invention.

FIG. 3 is a flowchart illustrating a preprocessing stage in the operating method of the MES detection device according to an embodiment of the invention.

FIG. 4 illustrates a training method of an AI algorithm used in the operating method of the MES detection device according to an embodiment of the invention.

FIG. 5 is a flowchart illustrating a procedure for changing an amplitude threshold and a duration threshold when MES are detected in the operating method of the MES detection device according to an embodiment of the invention.

FIG. 6 is a flowchart illustrating an operating method of the MES detection device with a vessel-position recognition function according to an embodiment of the invention.

### BEST MODE FOR CARRYING OUT THE INVENTION

The following detailed description refers to the accompanying drawings that illustrate specific embodiments in which the invention may be practiced. These embodiments are described in sufficient detail to enable those skilled in the art to practice the invention. Various embodiments may differ from each other but need not be mutually exclusive. For example, specific shapes, structures, and characteristics described herein may be modified and implemented from one embodiment to another without departing from the scope of the invention. Likewise, the positions or arrangements of individual components in each embodiment may be changed without departing from the scope of the invention. Therefore, the following detailed description is not intended to be limiting, and the scope of the invention is to be interpreted as covering the full scope defined by the claims and all equivalents thereof. Like reference numerals in the drawings denote identical or similar elements across various aspects.

Terms such as first and second may be used to describe various elements, but the elements are not limited by these terms, which are used solely to distinguish one element from another. For example, a first component may be termed a second component and vice versa without departing from the scope of the invention. The term “and/or” includes combinations of a plurality of associated items or any item among the plurality of associated items.

When an element is referred to as being “connected” or “coupled” to another, it can be directly connected or coupled to the other element, or intervening elements may be present. In contrast, when an element is referred to as being “directly connected” or “directly coupled” to another, there are no intervening elements present.

The terminology used herein is for the purpose of describing particular embodiments only and is not intended to limit the invention. The singular forms include the plural forms as well unless the context clearly dictates otherwise. The terms “comprise” and “have” and variations thereof specify the presence of stated features, integers, steps, operations, elements, components, or combinations thereof, but do not preclude the presence or addition of one or more other features, integers, steps, operations, elements, components, or combinations thereof.

As used herein, terms denoting degrees such as “about” and “substantially” are used to indicate that manufacturing and material tolerances inherent to the stated meaning are encompassed within the value, and to prevent unscrupulous use of exact or absolute values to misconstrue the disclosure. Phrases such as “step of ~ing” or “step of” do not mean “step for ~” unless expressly indicated otherwise.

As used herein, the term “module” refers to a unit implemented by hardware, a unit implemented by software, or a unit implemented by a combination of both. A single unit may be implemented using two or more hardware components, and two or more units may be implemented by a single hardware component.

Some of the operations or functions described as being performed by a terminal, device, or apparatus may instead be performed on a server connected thereto. Similarly, some operations or functions described as being performed by a server may be performed on a connected terminal, device, or apparatus.

Unless otherwise defined, all terms (including technical and scientific terms) used herein have the same meaning as commonly understood by one of ordinary skill in the art. Terms defined in commonly used dictionaries should be interpreted consistently with their meaning in the context of the relevant technology and the present disclosure, and not in an idealized or overly formal sense unless expressly defined herein.

Hereinafter, preferred embodiments of the invention will be described in more detail with reference to the drawings. To facilitate overall understanding, the same reference numerals are used for identical components, and repeated thereof are omitted.

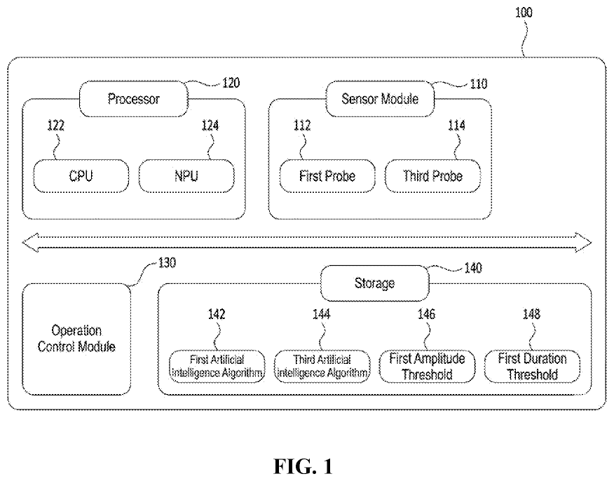

FIG. 1 is a block diagram of an MES detection device according to an embodiment of the invention.

Referring to FIG. 1, an MES detection device 100 may include a sensor module 110, a processor 120, an operation control module 130, and a storage 140. Depending on the embodiment, the device 100 may be implemented as a wearable device or a surgical device. Depending on the embodiment, the device 100 may operate in real time.

In some embodiments, the sensor module 110 may include a probe. One or more probes may be used, and each probe may be used for a particular purpose. For example, the sensor module 110 may include a first probe 112 and/or a third probe 114.

The first probe 112 may focus on analyzing ultrasonic echo signals generated in the subject's blood vessels to determine the presence of microemboli. Accordingly, it is possible to determine whether microemboli exist in the bloodstream in real time, which may contribute to detecting conditions like embolism and stroke at an early stage.

The third probe 114 may be used to identify the position of a target vessel. Vascular structures vary due to anatomical differences among patients, and accurate MES detection requires precise identification of vessel location. The third probe 114 may determine the relative positions of major vessels based on blood-flow velocity and direction, thereby assisting the first probe 112 in collecting signals at an optimal measurement target area. Employing multiple probes enables more precise hemodynamic analysis and reduces measurement errors common with single-probe systems. This allows medical personnel to receive more accurate diagnostic information and enables more effective patient health management through real-time monitoring.

In one embodiment, Doppler ultrasound analysis results may be provided via spectral Doppler ultrasonography. The spectral Doppler ultrasonography measures blood-flow velocity and enables quantitative evaluation. The analysis results may be provided as Doppler signal waveforms. The probe may output velocity and direction information of blood flow as a time-series waveform, enabling quantitative analysis of flow characteristics. In embodiments of the present invention, a frequency spectrum of the Doppler waveform is analyzed to extract patterns of blood-flow velocity variation within a vessel, and the vessel position is determined by searching for locations where specific flow patterns persist. By analyzing the waveform data, features such as blood flow velocity, pulsatility, and turbulence within the vessel can be precisely evaluated, thereby enabling more accurate localization of the vessel.

In one embodiment, Doppler ultrasound analysis results may be provided via color Doppler ultrasonography. The color Doppler ultrasound examination is a Doppler examination method that determines color based on the direction of blood flow and determines brightness based on the velocity of blood flow. The color Doppler examination may also provide an image of the measurement space. The probe may visually express the relative position of vessels within a particular space based on blood flow, and may explore vascular structures in 2D or 3D space relative to the probe. In embodiments, explored vessel positions may be expressed using color mapping or vector mapping, highlighting regions of high flow velocity to effectively identify specific vessels. Such image-based analysis results may be used by machine learning and AI models to automatically recognize vascular structures and select target vessels.

In some embodiments, MES are detected and target vessel positions are determined by combining spectral Doppler ultrasonography results and color Doppler ultrasonography results.

The labels “first probe 112” and “third probe 114” are used merely to distinguish functions and need not indicate physically different devices. The same probe may perform both MES detection and target vessel localization. Multiple probes serving the same purpose may also be mounted on a single MES detection device. For example, in order to increase the reliability of MES detection, multiple probes may be arranged for the same measurement purpose. In addition, to enhance the accuracy of vessel localization, multiple probes may be utilized to perform measurements from various angles.

According to one embodiment, the probe may be embedded in or coupled to the MES detection device 100.

The probe transmits ultrasonic signals and receives reflected signals, and measures blood flow velocity by analyzing the signals reflected from red blood cells in the bloodstream. The probe measures the blood flow velocity using the Doppler effect and includes an ultrasonic transducer embedded therein. In general, a frequency in the range of 1 MHz to 10 MHz is used. According to one embodiment, an ultrasonic frequency of 2 MHz may be used for measuring cerebral blood flow.

One or more probes may be installed on the device 100, and measurements may be performed using an optimal probe depending on relative position with respect to the target vessel. For example, two probes may be mounted on the left and right of the device 100, and MES measurements may be performed using the probe closer to the target vessel. For example, two probes may be mounted on the left and right of the device 100, and MES measurements may be performed using the probe closer to the target vessel. One probe may perform vessel localization and another may perform MES detection. Multiple probes may be utilized to collect signals from multiple angles, thereby improving measurement reliability. Data quality may be analyzed during measurement, and if the signal strength of a specific probe is low or distorted, the system may switch to another probe.

According to one embodiment, the MES detection device 100 may include a support or a fastening band. The support does not completely encircle the measurement target area but has a headband-like structure that is attached or fixed to only a portion of the measurement target area. The support may be relatively lightweight and designed to be stably mounted on a specific region. The fixing band may completely surround the site in a hairband form and be designed to fix the probe(s) more firmly; it may include elastic materials. Both the support and fixing band may include adjustable fasteners to allow probe repositioning and angle change during measurement.

According to one embodiment, the measurement target area may be head, neck, arm, wrist, chest, thigh, or ankle. The measurement target area is selected where major vessels with active flow are located, depending on measurement purpose and application environment. For the head, transcranial Doppler (TCD) ultrasound may measure cerebral blood flow and detect MES in the middle cerebral artery (MCA) and internal carotid artery (ICA). For the neck, carotid ultrasound may analyze carotid blood flow status and stenosis, and detect MES in the carotid before they travel to the brain. In the case of the arm and wrist regions, blood flow in peripheral arteries may be measured and utilized to evaluate the health status of systemic blood circulation. In particular, blood flow analysis can be performed in the radial artery and the brachial artery. Chest measurements may monitor flow in major arteries connected to the heart (e.g., aorta, subclavian). Thigh measurements may analyze lower-limb circulation via the femoral artery. In the case of the ankle region, blood flow measurement can be performed for detecting peripheral artery disease (PAD), and the ankle-brachial index (ABI) can be calculated to evaluate lower-limb blood circulation conditions. In addition, the measurement method for each region may vary depending on the arrangement and fixation manner of the probes. For example, in the head and neck regions, a support or fastening band may be used to stably maintain the position of the probe, whereas in the wrist or ankle regions, an elastic fastening band may be employed to minimize movement during measurement.

Probe position and angle may be adjusted by the operation control module 130 to enable optimal ultrasound measurement of the target vessel. If the target vessel moves or the subject moves, the probe direction may be automatically adjusted to maintain optimal measurement.

Target vessels may include the MCA, ICA, external carotid artery (ECA), common carotid artery (CCA), brachial artery, subclavian artery, internal thoracic artery, axillary artery, femoral artery, deep femoral artery, radial artery, ulnar artery, subclavian artery, aorta, tibial artery, or dorsalis pedis artery.

The target vessel position may be a position relative to the probe, expressed in a coordinate system such as Cartesian, polar, or cylindrical coordinates. When multiple probes are used, the position may be determined relative to one probe or a centroid of the probes.

In another embodiment, the positions and angles of the probes in the device 100 may be fixed at optimal settings for MES measurement.

The processor 120 may include a CPU 122 and an NPU 124. The CPU 122 may control the overall operation of the MES detection device 100. The CPU 122 may include a Single core or Multi-Core processors. The CPU 122 may process or execute programs and/or data stored in the storage 300. For example, by executing programs and/or modules stored in the storage 300, the CPU 122 may control the functions of the NPU 124. The CPU 122 may also implement a neural network model using implementation languages such as Java, C/C++, Python, or R, and frameworks based on Python, including TensorFlow, Keras, and PyTorch.

The processor 120 may store Doppler ultrasound measurement results obtained via the sensor module 110 in the storage. The processor 120 may adjust probe position and angle considering vessel position to acquire optimal signals.

The processor 120 may control execution of preprocessing of Doppler ultrasound measurement results acquired through the sensor module 110. The processor 120 may convert the acquired Doppler ultrasound measurement results into continuous frames to prevent data loss.

The processor 120 may apply a filtering technique to remove low-frequency or high-frequency noise and improve signal quality. The processor 120 may also transform the signal to emphasize specific patterns (e.g., amplitude or duration) so as to clarify the characteristics of microembolic signals (MES).

The processor 120 may analyze Doppler ultrasound measurement results to determine relative coordinates by identifying vessel locations. The processor 120 may automatically search for a target vessel by considering the flow velocity and direction of the blood. The processor 120 may also dynamically adjust the position and angle of the probe through the operation control module 130 based on the vessel search results to maintain an optimal measurement environment.

The processor 120 may extract feature vectors (e.g., amplitude, duration, frequency variation) from the signals for MES detection and evaluate the likelihood of microembolic signals. Applying a signal-analysis model based on the first AI algorithm 142, the processor 120 may classify signals and determine the presence of MES. Based on the analysis results, the processor 120 may evaluate detection confidence by comparing signal strength and patterns.

According to MES detection results, the processor 120 may update an amplitude threshold and/or a duration threshold used in preprocessing. Based on detected MES patterns, the processor 120 may dynamically adjust these thresholds.

In the preprocessing, the processor 120 may perform dynamic threshold variation. Where MES are determined to be present from the measurement results using the first AI algorithm 142, the processor 120 may lower a first amplitude threshold 146, raise a first duration threshold 148, or lower the first amplitude threshold 146 and raise the first duration threshold 148. The processor 120 may apply the adjusted first amplitude threshold 146, the adjusted second duration threshold 148, or both the adjusted first amplitude threshold 146 and the adjusted second duration threshold 148 in the step of obtaining preprocessed Doppler ultrasound measurement results by preprocessing subsequent Doppler ultrasound measurement results. The processor 120 can thereby modulate detection sensitivity by applying the updated thresholds to the subsequent measurement results.

The processor 120 may provide information on microemboli within the subject according to a determination as to whether MES are present. In one embodiment, the information may include at least one of: presence/absence of microemboli, detection location, detection frequency, MES signal strength, or a combination thereof. In one embodiment, the processor 120 may store such information in real time and output analysis results; the processor 120 may record data logs to enable trend analysis over time. In one embodiment, the processor 120 may provide information on microemboli within the subject in real time. In one embodiment, the information on the microemboli may be used for predicting cerebral infarction, myocardial infarction, or cancer metastasis.

In one embodiment, the processor 120 may perform machine learning of an artificial intelligence algorithm through the NPU 124. The NPU 124 is a processor designed to optimize machine learning operations including deep learning, and may serve to accelerate the training and execution of artificial intelligence algorithms. In particular, the NPU 124 may provide high computational efficiency for neural network models that require large-scale matrix operations and may support faster processing speed than the CPU 122 through parallel processing. In one embodiment, the NPU 124 may be used to perform training and optimized inference of a first artificial intelligence algorithm 142 and a third artificial intelligence algorithm 144 for analyzing Doppler ultrasound signals.

The processor 120 may be used not only for detecting MES from ultrasound signals but also for identifying target vessel positions. In the vessel localization process, the processor 120 may analyze probe signals to evaluate flow velocity and direction and may rapidly process large amounts of signal data to more precisely determine the relative position of vessels. Thus, the processor 120 may automatically select the probe closest to the target vessel and maintain optimal conditions to improve MES detection accuracy.

During signal preprocessing, the processor 120 may receive filtered Doppler ultrasound data as input, perform AI-based feature extraction and signal-pattern classification, and analyze signal characteristics more precisely. In addition, the processor 120 may be used during the training process of a machine learning model for vessel localization and MES detection. During real-time data analysis, the processor 120 may perform inference based on the trained model to rapidly determine the presence or absence of MES.

In one embodiment, depending on real-time needs and computational requirements, the CPU 122 and NPU 124 may be used in a suitable combination. That is, executing trained models to analyze signals and detect MES may be performed by the CPU alone; where real-time processing or large-scale parallel computation is required, the NPU 124 may be additionally utilized. This enables the present invention to enhance neural network-based MES detection accuracy and computational efficiency, thereby providing fast and effective diagnosis.

According to one embodiment, the NPU 124 may generate a neural network, train or learn the neural network, perform computations based on training data, and generate an information signal based on the computation results. The NPU 124 may also retrain the neural network. The NPU 124 is not limited to its name and may include any processor configured to execute neural networks, such as a GPU or a TPU.

Depending on the embodiment, neural-network models may include CNNs (e.g., GoogLeNet, AlexNet, VGGNet), R-CNN, RPN, RNN, stacking-based deep neural networks (S-DNN), state-space dynamic neural networks (S-SDNN), deconvolution networks, deep belief networks (DBN), restricted Boltzmann machines (RBM), fully convolutional networks, LSTM networks, classification networks, and the like. Reinforcement-learning models may include DQN, DDQN, dueling DQN, DDPG, DDDQN, etc.

According to one embodiment, the processor 120 may use a separate storage 140 for storing programs corresponding to neural network models. The processor 120 may further include separate intellectual property (IP) blocks for processing a large number of computations required to operate the neural network. For example, the separate IP blocks may further include a graphical processing unit (GPU) or an accelerator configured to perform specific operations at high speed.

According to one embodiment, the operation control module 130 may control the movement of the MES detection device 100. For example, the operation control module 130 may control configurations for moving the MES detection device 100 according to the position of a target vessel, such as the shape of the support, the length of the support, the tightness of the fastening band, the position of the probe on the support and/or the fastening band, and the angle of the probe.

In one embodiment, the operation control module 130 may adjust the position and angle of the first probe 112 or the third probe 114 so that the first probe 112 or the third probe 114 is directed toward the target vessel position. In one embodiment, the processor 120 may adjust the position and angle of the first probe 112 or the third probe 114 through the operation control module 130, based on information regarding the position of the target vessel. In one embodiment, the operation control module 130 may adjust the shape of the support, the length of the support, or the tightness of the fastening band.

According to one embodiment, the storage 140 may serve as a storage medium for storing data and may store an operating system (OS), various programs, and various types of data. For example, the storage 140 may correspond to non-volatile memory. The storage 140 may include a read-only memory (ROM), flash memory, phase-change RAM (PRAM), magnetic RAM (MRAM), resistive RAM (RRAM), or ferroelectric RAM (FRAM). According to one embodiment, the storage 140 may be implemented as a hard disk drive (HDD), a solid-state drive (SSD), or the like.

According to one embodiment, the storage 140 may store a first artificial intelligence algorithm 142 and a third artificial intelligence algorithm 144. The first artificial intelligence algorithm 142 may be a neural network model for determining the presence of microembolic signals (MES) from preprocessed Doppler ultrasound measurement results. The first artificial intelligence algorithm 142 may receive the preprocessed Doppler ultrasound measurement results as input, analyze MES patterns, and provide detection results. The third artificial intelligence algorithm 144 may be used to search for the location of a target vessel and determine relative coordinates, automatically locating the target vessel and setting the optimal probe position. These artificial intelligence algorithms may be stored as pre-trained models or may be designed to be continuously updated when new data are added.

According to one embodiment, the storage 140 may store a first amplitude threshold 146 and a first duration threshold 148. The first amplitude threshold 146 may serve as a reference value for removing noise based on the amplitude of a signal and may be used to identify, as MES candidates, signals having amplitudes exceeding a certain level in Doppler ultrasound data. The first duration threshold 148 may serve as a reference for determining that only signals lasting longer than a specific time are MES, thereby filtering out transient variations and enabling reliable MES detection. These thresholds may be dynamically updated based on the data stored in the storage 140 and real-time analysis results and may also be adjusted according to user settings to improve detection accuracy.

In addition, the storage 140 may store initial training datasets, user-defined threshold settings, patient-specific measurement histories, raw Doppler ultrasound signal data, and analyzed feature vectors.

Through this configuration, the reliability of MES detection results can be verified by comparing them with previously measured data, and long-term monitoring of changes in blood flow patterns of a specific patient can be supported. Furthermore, the storage 140 may be linked to a cloud-based storage system, enabling real-time diagnostic data sharing through connection with remote medical systems, or allowing medical professionals to retrieve past data for in-depth analysis when necessary.

In one embodiment, the MES detection device may include a sensor module comprising a first probe, a storage configured to store a first artificial intelligence algorithm, and a processor configured to obtain a first Doppler ultrasound measurement result by using the first probe on a measurement target area of a subject, to preprocess the first Doppler ultrasound measurement result to obtain a preprocessed first Doppler ultrasound measurement result, to determine the presence of microembolic signals (MES) from the preprocessed first Doppler ultrasound measurement result by using the first artificial intelligence algorithm, and to provide information on microemboli within the subject according to whether the MES are present.

In one embodiment, the MES detection device may include a sensor module comprising a first probe, a storage configured to store a first artificial intelligence algorithm and a third artificial intelligence algorithm, and a processor configured to obtain a third Doppler ultrasound measurement result by using a third probe on a measurement target area of a subject, to preprocess the third Doppler ultrasound measurement result to obtain a preprocessed third Doppler ultrasound measurement result, to obtain the position of the target vessel from the preprocessed third Doppler ultrasound measurement result by using the third artificial intelligence algorithm, to obtain a first Doppler ultrasound measurement result by directing the first probe toward the target vessel position of the measurement target area of the subject, to preprocess the first Doppler ultrasound measurement result to obtain a preprocessed first Doppler ultrasound measurement result, to determine the presence of microembolic signals (MES) from the preprocessed first Doppler ultrasound measurement result by using the first artificial intelligence algorithm, and to provide information on microemboli within the subject according to whether the MES are present.

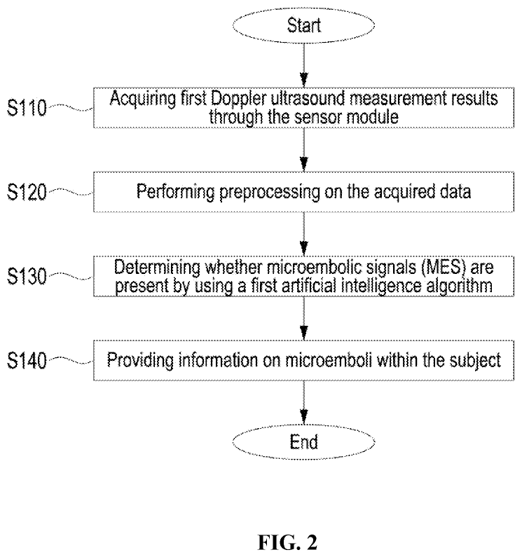

FIG. 2 is a flowchart illustrating an operation method of the MES detection device according to an embodiment of the present invention.

The operation method of the MES detection device of the present invention may include acquiring a first Doppler ultrasound measurement result through a sensor module S110, performing preprocessing of the acquired data S120, determining whether MES are present by using a first artificial intelligence algorithm S130, and providing information on microemboli within the subject S140. Such an operation method may be performed in real time.

In step S110, in which a first Doppler ultrasound measurement result is acquired through the sensor module, the first probe is applied to a measurement target site and is used for MES detection.

In step S120, in which preprocessing of the acquired data is performed, the data undergo a preprocessing procedure so that the first artificial intelligence algorithm can be effectively utilized. In one embodiment, the processor 120 performs noise removal and signal normalization.

In step S130, in which the presence of MES is determined by using the first artificial intelligence algorithm, the first artificial intelligence algorithm may detect MES from the preprocessed Doppler ultrasound signals. In one embodiment, the first artificial intelligence algorithm may be trained based on MES clinical data. The MES clinical data may be clinical data labeled to indicate whether MES are present in Doppler ultrasound measurement results. The first artificial intelligence algorithm may be trained to match the preprocessed Doppler ultrasound measurement results (for example, second Doppler ultrasound measurement results) with the labeled presence or absence of MES based on MES clinical data. In one embodiment, during the process of training the first artificial intelligence algorithm, the preprocessed second Doppler ultrasound measurement results obtained by preprocessing Doppler ultrasound measurement results based on MES clinical data may be used as training data, and the clinical data regarding the presence or absence of MES may be used as labeling data. In one embodiment, features of MES (for example, signal amplitude, frequency variation, duration, and the like) may be directly extracted, and machine learning may be performed based on such features.

In step S140, in which information on microemboli within the subject is provided, information on microemboli within the subject may be provided according to whether MES are present. According to one embodiment, the information on microemboli within the subject may include at least one of the presence of microemboli, the detection frequency of microemboli, the signal intensity of MES, or a combination thereof. Dynamic thresholds may also be applied as necessary. For example, when MES are detected, the first amplitude threshold 146 and the first duration threshold 148 may be dynamically changed, and the updated thresholds may be applied in subsequent detection processes. Additionally, the detected data may be stored in the storage 140 and used as data for long-term patient monitoring and further analysis.

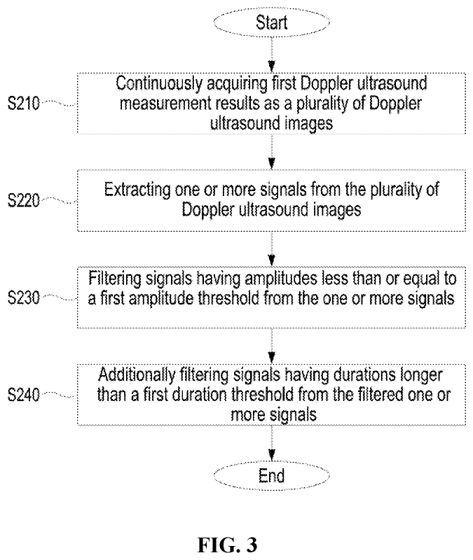

FIG. 3 is a flowchart illustrating a preprocessing step in the operation method of the MES detection device according to an embodiment of the present invention.

In the operation method of the MES detection device of the present invention, the step S120 of performing preprocessing of the acquired data may include continuously acquiring first Doppler ultrasound measurement results as a plurality of Doppler ultrasound images S210, extracting one or more signals from the plurality of Doppler ultrasound images S220, filtering signals having amplitudes equal to or lower than a first amplitude threshold from the one or more signals S230, and/or further filtering signals having durations longer than a first duration threshold from the filtered one or more signals S240.

In step S210, in which the first Doppler ultrasound measurement results are continuously acquired as a plurality of Doppler ultrasound images, the process of acquiring blood flow signals from a measurement target area by using a probe may be performed. In the embodiment of the present invention, the first probe may be used for MES detection and may measure continuous blood flow signals. During the signal acquisition process, a plurality of Doppler ultrasound images may be continuously collected, and data may be acquired in an overlapping manner so that the signals are continuously maintained without data loss. The Doppler ultrasound signals may be captured as continuous image frames and subdivided in an overlapping state so that no signal is omitted. In one embodiment, the Doppler ultrasound signals may be divided into time segments of 100 ms to 200 ms, and more than 50% overlap may be applied between segments to prevent loss of continuous data. In another embodiment, the divided Doppler ultrasound signals may have 10% to 90% or more overlap between segments to prevent continuous data loss. Through this configuration, continuous detection can be achieved without signal loss even when MES exist across the boundary of a single frame.

In step S220, in which one or more signals are extracted from the plurality of Doppler ultrasound images, the continuously acquired signals may be arranged in chronological order, and frames may be merged based on overlapping data with previous segments to extract one or more signals in which the flow is maintained. The signal amplitude and duration may be standardized so that consistent analysis can be performed in all measurement environments. In one embodiment, Fourier Transform may be utilized to convert the signals into a frequency domain, and unnecessary noise may be removed by analyzing the frequency spectrum. In particular, because MES signals exhibit characteristic patterns in specific frequency bands, MES candidate signals can be more clearly distinguished through signal filtering and frequency analysis.

In step S230, in which signals having amplitudes equal to or lower than the first amplitude threshold are filtered from the one or more signals, a high-pass filter may be applied to reduce low-frequency components and external interference included in the signal. In one embodiment, only signals having an amplitude that is 1 to 6 dB higher than the background blood flow velocity may be retained to separate MES candidate signals. In another embodiment, only signals having an amplitude 3 dB higher than the background blood flow velocity may be retained to separate MES candidate signals. In one embodiment, signals having amplitudes less than 3 dB relative to the background blood flow velocity may be filtered out to isolate MES candidate signals. In one embodiment, the first amplitude threshold may be 1 to 6 dB. In another embodiment, the first amplitude threshold may be 4 to 5 dB. In another embodiment, the first amplitude threshold may be 3 dB.

In step S240, in which signals having durations longer than the first duration threshold are further filtered from the filtered one or more signals, when the duration of a signal is maintained for a specific time (for example, 0.5 to 1 second) or longer, the signal may be removed because it is likely to correspond to a long-term blood-flow change or noise. Conversely, short transient signals may be retained because they are more likely to represent MES. In one embodiment, the first duration threshold may be 0.5 to 2 seconds. In another embodiment, the first duration threshold may be 0.5 to 1 second. In another embodiment, the first duration threshold may be 100 to 1000% of the average signal duration of MES. In one embodiment, the first duration threshold may be 100 to 500%, 100 to 300%, or 100 to 200% of the average signal duration of MES. In one embodiment, the average signal duration of MES may be 0.5 to 1 second, and in another embodiment, it may be 0.75 second. In one embodiment, a multi-frame comparison method may be applied during the signal filtering process to improve reliability by evaluating whether an MES candidate signal repeatedly occurs within a specific time interval. Additionally, a correction process may be performed so that key features (e.g., amplitude, duration, frequency variation, etc.) are preserved even after filtering. Finally, the filtered signals may be output in an analyzable form, and the analysis results may be stored. In the embodiment of the present invention, the preprocessed signals are stored in the processor and used as input data for subsequent artificial intelligence analysis.

In one embodiment, in the operation method of the MES detection device, the steps related to performing preprocessing of the acquired data S120—namely, the step S210 of continuously acquiring first Doppler ultrasound measurement results as a plurality of Doppler ultrasound images, the step S220 of extracting one or more signals from the plurality of Doppler ultrasound images, the step S230 of filtering signals having amplitudes equal to or lower than a first amplitude threshold from the one or more signals, and the step S240 of further filtering signals having durations longer than a first duration threshold from the filtered one or more signals—may each be independently used to perform preprocessing or may be used in combination thereof.

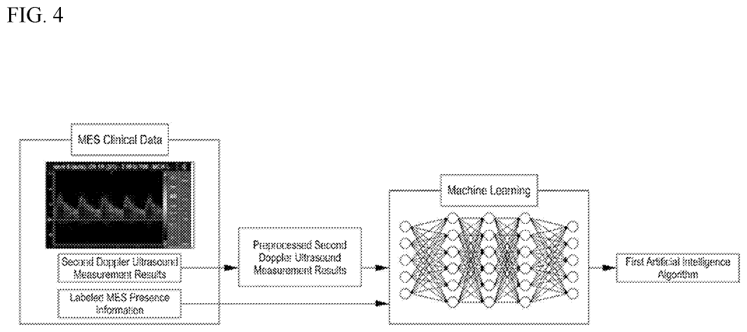

FIG. 4 illustrates a training method of an artificial intelligence algorithm used in the operation method of the MES detection device according to an embodiment of the present invention.

The first artificial intelligence algorithm may be trained to match the preprocessed second Doppler ultrasound measurement results based on MES clinical data with labeled MES presence data.

In one embodiment, training data may be generated by preprocessing raw signals measured by a probe. In one embodiment, the preprocessing process may be the same as the step S120 of performing preprocessing of the acquired data from the first Doppler ultrasound measurement results. That is, the process may include continuously acquiring second Doppler ultrasound measurement results as a plurality of Doppler ultrasound images, extracting one or more signals from the plurality of Doppler ultrasound images, filtering signals having amplitudes equal to or lower than a second amplitude threshold, and further filtering signals having durations longer than a second duration threshold from the filtered one or more signals. The second amplitude threshold may be the same as the first amplitude threshold, and the second duration threshold may be the same as the first duration threshold. Signals obtained by preprocessing Doppler ultrasound measurement results based on clinical data may be used as training data. The training data may be divided into specific time segments (e.g., units of 100 to 200 ms) and may partially overlap to maintain signal continuity. Each signal may have noise removed through frequency analysis and filtering and may be normalized to emphasize MES characteristics. The training data may include signal features such as amplitude, frequency variation, and duration. Because MES signals have relatively higher amplitudes than background blood flow, data may be organized based on signal strength. Methods such as Fourier Transform may be applied to analyze how signals change within specific frequency bands. Since MES signals occur in short instances, they may be classified based on specific duration ranges. The training data may include Doppler ultrasound data collected in various environments and may be categorized into dataset according to measurement target areas (head, neck, arm, etc.) and vessel types (middle cerebral artery, carotid artery, femoral artery, etc.).

In one embodiment, labeling data may be ground truth data corresponding to the training data and may include information indicating whether each Doppler ultrasound signal contains an MES. In an embodiment of the present invention, signals may be labeled based on clinical data on MES presence, allowing the machine learning model to learn the correct pattern. The labeling data may be constructed based on clinical studies and expert medical analysis and may classify Doppler ultrasound signals as actual MES, normal blood flow, or noise (artifact). Signals determined to be MES may be further grouped according to signal intensity, frequency pattern, and duration and may be clearly distinguished from non-MES signals. To prevent data imbalance during neural network training, the labeled data may be augmented and variously transformed through data augmentation processes. In an embodiment of the present invention, labeling may be performed using binary classification or multi-class classification. In binary classification, each Doppler ultrasound signal may be classified as either MES or non-MES. In multi-class classification, the severity of MES signals may be distinguished, or specific signal patterns may be subdivided for classification. The MES clinical data may include Doppler ultrasound examination results collected from neurological and cardiological studies, patient data measured in hospitals, publicly available medical datasets, and proprietary datasets. These data may be labeled to distinguish MES signals from normal blood flow signals and may include feature vectors such as signal amplitude, frequency variation, and duration.

In one embodiment, the neural network model may utilize a convolutional neural network (CNN) or a long short-term memory (LSTM) network. The CNN model may be used to learn MES features and vessel localization features from preprocessed Doppler ultrasound image data. The LSTM model may be applied to learn temporal variation patterns of signals and to reflect signal continuity. If necessary, a hybrid model combining CNN and LSTM may be employed. During the model training process, a cross-entropy loss function may be used as a loss function to evaluate learning performance. Algorithms such as Adam or RMSprop may be employed as optimizers to adjust learning speed and derive optimal results. The trained model may be evaluated using validation and test datasets. Model performance may be assessed based on metrics such as accuracy, precision, recall, and F1-score.

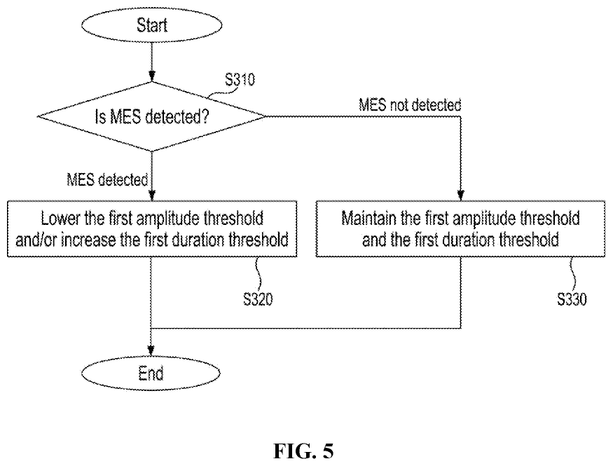

FIG. 5 is a flowchart illustrating a procedure for changing the amplitude threshold and duration threshold according to MES detection in the operation method of the MES detection device according to an embodiment of the present invention.

The operation method of the MES detection device of the present invention may include determining whether MES are detected S310 after determining the presence of MES by using the first artificial intelligence algorithm S130 or providing information on microemboli within the subject S140, and, if MES are detected, lowering the first amplitude threshold or increasing the first duration threshold S320, and, if MES are not detected, maintaining the first amplitude threshold and the first duration threshold S330.

In step S310, in which it is determined whether MES are detected, the process of analyzing Doppler ultrasound signals to check whether MES have been detected may be performed. In an embodiment of the present invention, the first artificial intelligence algorithm performs signal analysis, and when MES are detected a certain number of times or more within a specific period, it may be determined that MES occurrences appear as a continuous pattern. It may be analyzed whether the detected MES signals are single events or continuously occurring patterns. If MES are repeatedly detected over a certain period, the amplitude and duration thresholds may be automatically adjusted to reflect the signal characteristics. Conversely, if MES are not detected for a certain time period, the thresholds may be restored to their default values.

In step S320, in which the first amplitude threshold is lowered or the first duration threshold is increased when MES are detected, the process of determining whether to maintain or adjust the currently set thresholds (the first amplitude threshold and the first duration threshold) may be performed. When a new MES pattern is detected, it may be evaluated whether the existing thresholds are appropriate for optimizing MES detection. If the existing thresholds are too high and may miss some MES signals, the amplitude threshold may be lowered or the duration threshold may be increased to enhance detection sensitivity. Conversely, if it is necessary to reduce false positives (noise signals), the amplitude threshold may be increased or the duration threshold may be extended. In an embodiment of the present invention, the threshold adjustment range may be dynamically controlled; for example, the amplitude threshold may be adjusted within a range of 1 to 6 dB, and the duration threshold may be adjusted within a range of 0.5 to 2 seconds.

The adjusted thresholds may be stored in the storage 140 and subsequently applied in the following analysis process. For newly collected Doppler ultrasound signals, the updated thresholds may be applied to optimize detection sensitivity. If MES are not detected for a certain period, the thresholds may be restored to their default values. In an embodiment of the present invention, by dynamically optimizing the thresholds based on previous analysis results, the system can maintain MES detection accuracy while flexibly responding to variations in signals.

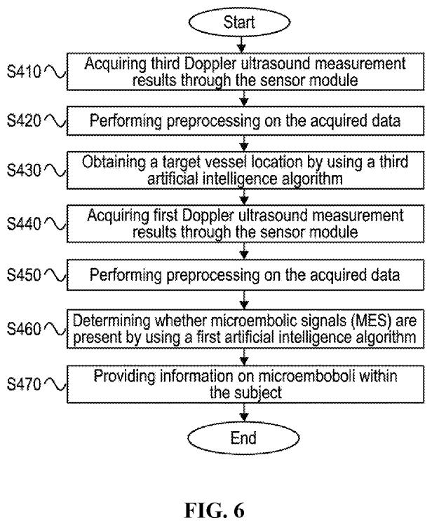

FIG. 6 is a flowchart illustrating an operation method of the MES detection device having a vessel localization function according to an embodiment of the present invention.

Another operation method of the MES detection device according to an embodiment of the present invention may include acquiring third Doppler ultrasound measurement results through a sensor module S410, performing preprocessing of the acquired data S420, obtaining the position of a target vessel by using a third artificial intelligence algorithm S430, acquiring first Doppler ultrasound measurement results through the sensor module S440, performing preprocessing of the acquired data S450, determining the presence of MES by using a first artificial intelligence algorithm S460, and providing information on microemboli within the subject S470.

In step S410, in which the third Doppler ultrasound measurement results are acquired through the sensor module, a process of acquiring signals for vessel search by using a probe may be performed. In one embodiment, a third probe may analyze blood flow patterns and perform a role of searching for the position of a target vessel. Because vascular structures vary among individuals, it is necessary to precisely locate the target vessel. The probe may transmit multiple-frequency signals to measure the velocity and direction of blood flow, and when a constant flow pattern is detected in a specific vessel, the corresponding location may be set as the identified vessel position. In this step, data may be accumulated over a certain period to maintain signal continuity and used in subsequent analysis.

In one embodiment, the Doppler ultrasound analysis results may be provided as spectral Doppler ultrasonography. Spectral Doppler ultrasonography is a diagnostic method that measures blood flow velocity and evaluates it graphically and numerically. The results of spectral Doppler ultrasonography may also be provided in the form of Doppler signal waveforms. In one embodiment, based on the acquired waveform data, variations in blood flow velocity may be analyzed, and the location at which a specific flow pattern remains constant may be identified to determine the vessel position.

In one embodiment, the Doppler ultrasound analysis results may be provided as color Doppler ultrasonography. Color Doppler ultrasonography is a diagnostic method that assigns colors according to the direction of blood flow and determines brightness according to flow velocity. Color Doppler ultrasonography may also provide an image of the measurement space. The probe may visually represent the relative position of a vessel within a given space based on the blood flow pattern and may perform a function of exploring vascular structures in two-dimensional (2D) or three-dimensional (3D) space relative to the ultrasound probe.

In one embodiment, the third Doppler ultrasound measurement results may be obtained from spectral Doppler ultrasonography results, color Doppler ultrasonography results, or a combination thereof.

In step S420, in which preprocessing of the acquired data is performed, preprocessing of the obtained third Doppler ultrasound measurement results may be performed to prepare for vessel position analysis. In the embodiment of the present invention, various signal-processing and normalization techniques may be applied to improve the accuracy of vessel localization, aiming to maintain data quality and enhance analyzability. In one embodiment, the preprocessing procedure of the third Doppler ultrasound measurement results may be the same as the preprocessing procedure of the first Doppler ultrasound measurement results.

In one embodiment, the third Doppler ultrasound measurement results may be provided as image data visually representing the direction and velocity of blood flow, and various preprocessing procedures may be performed to analyze such data. The preprocessing of color Doppler ultrasound image data may include steps such as image quality enhancement, color correction, spatial alignment, and feature extraction, etc., thereby enabling more accurate vessel position analysis and optimization of signal processing for MES detection.

Because color Doppler ultrasound examination results may contain various types of noise in medical imaging environments, filtering and image correction processes may be performed to improve signal quality. A Gaussian filter or a median filter may be applied to remove high-frequency noise occurring in ultrasound images and to emphasize continuous blood flow patterns. Binary thresholding and contrast enhancement may be applied to clearly distinguish blood flow from surrounding tissues and improve analytical accuracy. A spatial smoothing technique may also be applied to maintain uniformity of blood flow patterns while minimizing unnecessary background noise.

Because color Doppler ultrasound examination results are characterized by colors assigned according to the direction of blood flow and brightness varying according to flow velocity, color correction may be performed to facilitate analysis. An “RGB to HSV color space conversion” may be applied to convert the color space of ultrasound images from the red-green-blue (RGB) color space to the hue-saturation-value (HSV) color space for more effective color analysis. Color mapping analysis may be performed to emphasize blood flow patterns of specific colors, thereby allowing clearer identification of the flow of specific vessels. Channel separation may be applied to separate individual color channels for analyzing blood flow direction based on color information.

Because color Doppler ultrasound images may be spatially distorted depending on the movement of the probe and the ultrasound measurement angle, alignment and correction processes for analysis may be performed. A geometric transformation may be applied to correct image distortion caused by changes in probe position. A region of interest (ROI) may be set so that only specific vessels are analyzed. Multi-frame alignment may be performed based on continuous image data to track vessel positions and compensate for image loss in specific frames.

In the preprocessed color Doppler ultrasound examination results, a process of analyzing characteristics of blood flow and extracting features usable for MES detection may be performed. Blood-flow-velocity-based feature analysis may quantify blood flow velocity based on changes in brightness (intensity) and hue in color Doppler images and may analyze velocity variation patterns to detect abnormal turbulence or vascular stenosis. Vessel-structure-based feature analysis may identify vessel shapes within the image, extract vessel diameters and orientations, detect continuous vascular patterns in color Doppler images, and optimize the location of specific vessels. Blood flow direction analysis and MES detection may evaluate MES likelihood and classify signal characteristics when abrupt changes in flow direction or multiple signal patterns within a specific vessel are detected.

The preprocessed color Doppler ultrasound examination results may be stored in the storage 140 and subsequently utilized in vessel localization and MES detection algorithms.

When spectral Doppler ultrasound examination results (waveform data) and color Doppler ultrasound examination results (image data) are combined, data alignment may be performed to facilitate analysis. Based on blood flow velocity and frequency variation, the signal intensity may be normalized to minimize deviations that may occur depending on ultrasound measurement conditions. For image-based data (color Doppler ultrasound examination results), color correction and sharpening may be applied, while for waveform data (spectral Doppler ultrasound examination results), high-frequency and low-frequency components may be adjusted to minimize noise.

Filtering techniques may be applied to remove external interference and environmental noise that may occur during the measurement process. High-pass filtering and low-pass filtering may be applied to improve signal quality. In spectral Doppler ultrasound signals, the signal-to-noise ratio (SNR) may be optimized to retain only vascular signals while removing background signals.

When some signals are lost or the data intervals are inconsistent in specific segments, interpolation techniques may be applied to complement the data. Signals may be adjusted to maintain consistent flow patterns based on blood flow velocity variations so that the relative position of vessels can be more clearly identified. Multi-frame analysis may be performed to compensate for missing vessel information in a single frame using consecutive frame data.

In this process, color Doppler ultrasound image data and spectral Doppler ultrasound waveform data may be combined to comprehensively analyze vascular structures and blood flow velocity patterns.

In step S430, in which the position of a target vessel is obtained by using the third artificial intelligence algorithm, a process of obtaining the final position of the target vessel may be performed based on preprocessed candidate vessel location data. In an embodiment of the present invention, analysis using a neural network model may be performed considering blood flow velocity, direction, and flow patterns, and it may be determined whether a specific vessel is optimized for MES detection.

In an embodiment of the present invention, the third artificial intelligence algorithm may apply a neural network model trained for Doppler ultrasound-based vessel localization.

As training data, spectral Doppler ultrasound examination results and color Doppler ultrasound examination results collected based on clinical data may be included. In one embodiment, fourth Doppler ultrasound measurement results may be used as training data. The dataset may include location information of target vessels (such as the middle cerebral artery, carotid artery, femoral artery, etc.) and blood flow signal patterns collected from those vessels. The dataset may include vascular data measured at various body parts so that vessel localization can be performed accurately for a specific region.

As an analysis method, image analysis based on a convolutional neural network (CNN) or a long short-term memory (LSTM) model may be applied. The CNN may learn color Doppler ultrasound examination data to automatically recognize vascular structures, and the LSTM model may analyze spectral Doppler ultrasound waveform data to learn continuous variations in blood flow velocity. By combining these two analysis results, the neural network model may determine whether a specific vessel is an appropriate target for MES detection.

Labeling data may include optimal MES measurement vessel locations based on cerebrovascular anatomical data. The relative position of the vessel may be determined with reference to the probe, and various coordinate systems such as Cartesian coordinates, polar coordinates, or cylindrical coordinates may be applied to represent the relative position. In one embodiment, the cerebrovascular anatomical data may be used to optimize vessel localization and MES detection based on Doppler ultrasound analysis and may include information on major cerebral vascular structures and blood flow patterns. Such data may be constructed based on anatomical characteristics such as the spatial arrangement of cerebral vessels, blood flow velocity and direction, and vessel diameter, enabling accurate identification of specific vessels and evaluation of blood flow conditions. In one embodiment, the cerebrovascular anatomical data may include information on major vessels such as the middle cerebral artery (MCA), anterior cerebral artery (ACA), posterior cerebral artery (PCA), basilar artery (BA), vertebral artery (VA), and carotid arteries (ICA, CCA, ECA). The data may include datasets collected from various age groups and pathological conditions (for example, stroke, vascular stenosis, etc.) to reflect individual anatomical variations and may provide normal blood flow velocity ranges for each vessel, thereby improving the reliability of Doppler ultrasound analysis.

After the vessel position is determined, the third artificial intelligence algorithm may select the optimal target vessel by considering signal intensity and flow patterns. To select the vessel suitable for MES detection, the blood flow intensity and pattern of the target vessel may be evaluated. If the blood flow velocity within a vessel is too low or irregular, another vessel may be selected to increase measurement reliability. The vessel position may be updated in real time and automatically corrected when the measurement environment changes.

The obtained vessel position data and the third artificial intelligence algorithm may be stored in the storage 140 and referenced in subsequent MES detection processes. By storing the relative position of the vessel, the same measurement region can maintain optimal measurement conditions during repeated examinations. When MES detection is subsequently performed, the probe may be automatically adjusted based on the previously identified vessel position.

The steps of acquiring the first Doppler ultrasound measurement results through the sensor module S440, performing preprocessing of the acquired data S450, determining the presence of MES by using the first artificial intelligence algorithm S460, and providing information on microemboli within the subject S470 may be identical to steps S110 through S140 of FIG. 1. In the step of providing information on microemboli within the subject S470, the information on microemboli may include at least one of the presence of microemboli, the detection location of microemboli, the detection frequency of microemboli, the signal intensity of the MES, or a combination thereof.

The embodiments of the present invention described above may be implemented in the form of program instructions executable through various computer components and may be recorded on a computer-readable recording medium. In one embodiment, a non-transitory computer-readable recording medium storing a computer program for executing the MES detection method may be provided. The computer-readable recording medium may include program instructions, data files, or data structures, either alone or in combination. The program instructions recorded on the computer-readable recording medium may be specifically designed and configured for the present invention, or may be code that is well known and available to those skilled in the field of computer software. Examples of the computer-readable recording medium include magnetic media such as hard disks, floppy disks, and magnetic tapes; optical recording media such as CD-ROMs and DVDs; magneto-optical media such as floptical disks; and hardware devices such as ROM, RAM, and flash memory that are specially configured to store and execute program instructions. Examples of the program instructions include not only machine code generated by a compiler but also high-level language code executable by a computer using an interpreter or the like. Hardware devices may be changed into one or more software modules to perform the processes according to the present invention, and vice versa.

Although the present invention has been described above with reference to specific components, limited embodiments, and drawings, such descriptions are provided only to promote a more comprehensive understanding of the invention. The present invention is not limited to the above embodiments, and those skilled in the art to which the invention pertains may make various modifications and alterations based on the disclosed content.

Therefore, the scope of the present invention should not be limited by the foregoing embodiments, and all ranges equivalent to or modified from the claims below shall be construed as falling within the scope of the present invention.

### DESCRIPTION OF THE REFERENCE NUMERALS

100: MES Detection Device
110: Sensor Module
112: First Probe
114: Second Probe
120: Processor
122: CPU
124: NPU
130: Operation Control Module
140: Storage
142: First Artificial Intelligence Algorithm
144: Third Artificial Intelligence Algorithm
146: First Amplitude Threshold
148: First Duration Threshold

What is claimed is:

1. A method for providing information on whether microemboli exist within a subject using Doppler ultrasound, the method comprising:
acquiring, by using a first probe on a measurement target area of the subject, first Doppler ultrasound measurement results;
preprocessing, by a processor, the first Doppler ultrasound measurement results to obtain preprocessed first Doppler ultrasound measurement results;
determining, by the processor, whether microembolic signals (MES, Microembolic Signal) are present in the preprocessed first Doppler ultrasound measurement results by using a first artificial intelligence algorithm; and
providing, by the processor, information on microemboli within the subject according to whether the MES are present wherein the preprocessing the first Doppler ultrasound measurement results to obtain the preprocessed first Doppler ultrasound measurement results comprises:
continuously acquiring, by the processor, the first Doppler ultrasound measurement results as a plurality of Doppler ultrasound images; and
obtaining, by the processor, preprocessed first Doppler ultrasound measurement results from the plurality of Doppler ultrasound images,
wherein the plurality of Doppler ultrasound images overlap each other so that no Doppler ultrasound signal is omitted between the images wherein obtaining the preprocessed first Doppler ultrasound measurement results from the plurality of Doppler ultrasound images comprises:
extracting, by the processor, one or more signals from the plurality of Doppler ultrasound images;
filtering, by the processor, signals having amplitudes less than or equal to a first amplitude threshold from the one or more signals; and
obtaining, by the processor, preprocessed first Doppler ultrasound measurement results from the one or more signals from which signals having amplitudes less than or equal to the first amplitude threshold are filtered
wherein obtaining the preprocessed first Doppler ultrasound measurement results from the one or more signals from which signals having amplitudes less than or equal to the first amplitude threshold are filtered comprises:
additionally filtering, by the processor, signals having durations longer than a first duration threshold from the filtered one or more signals; and
obtaining, by the processor, preprocessed first Doppler ultrasound measurement results from the one or more signals from which signals having durations longer than the first duration threshold are further filtered
wherein, when it is determined by the first artificial intelligence algorithm that MES are present in the first Doppler ultrasound measurement results,
by the processor, lowering the first amplitude threshold, or increasing the first duration threshold, or both lowering the first amplitude threshold and increasing the first duration threshold; and
in subsequently performed preprocessing first Doppler ultrasound measurement results to obtain preprocessed first Doppler ultrasound measurement results, applying the varied first amplitude threshold, the varied first duration threshold, or the varied first amplitude threshold and the varied first amplitude threshold.

2. The method of claim 1, wherein providing information by the processor on microemboli within the subject according to whether the MES are present is performed in real time.

3. The method of claim 1, wherein the measurement target area comprises a head, neck, arm, wrist, chest, thigh, or ankle.

4. The method of claim 1, wherein the first artificial intelligence algorithm is trained to match preprocessed second Doppler ultrasound measurement results with labeled MES presence data based on MES clinical data.

5. The method of claim 1, wherein the information on microemboli within the subject is used for prediction of cerebral infarction, myocardial infarction, or cancer metastasis.

6. The method of claim 1, wherein the method of providing information on whether microemboli exist within the subject is performed by a wearable device or a surgical device.

7. A method for providing information on whether microemboli exist within a subject using Doppler ultrasound, the method comprising:
acquiring, by using a first probe on a measurement target area of the subject, first Doppler ultrasound measurement results;
preprocessing, by a processor, the first Doppler ultrasound measurement results to obtain preprocessed first Doppler ultrasound measurement results;
determining, by the processor, whether microembolic signals (MES, Microembolic Signal) are present in the preprocessed first Doppler ultrasound measurement results by using a first artificial intelligence algorithm; and
providing, by the processor, information on microemboli within the subject according to whether the MES are present
wherein acquiring the first Doppler ultrasound measurement results comprises acquiring, by using the first probe, the first Doppler ultrasound measurement results directed toward a target vessel at the measurement target area of the subject
wherein the target vessel location is obtained by:
acquiring, by using a third probe, third Doppler ultrasound measurement results for the measurement target area of the subject;
preprocessing, by the processor, the third Doppler ultrasound measurement results to obtain preprocessed third Doppler ultrasound measurement results; and
obtaining, by the processor, the target vessel location from the preprocessed third Doppler ultrasound measurement results by using a third artificial intelligence algorithm.

8. The method of claim 7, wherein the target vessel comprises at least one of a middle cerebral artery, internal carotid artery, external carotid artery, common carotid artery, brachial artery, brachiocephalic artery, internal thoracic artery, axillary artery, femoral artery, or superficial femoral artery.

9. The method of claim 7, wherein the target vessel location is a relative position with respect to the first probe or the third probe.

10. The method of claim 7, wherein an operation control module adjusts a position and angle of the first probe so that the first probe is directed toward the target vessel location.

11. The method of claim 7, wherein the third artificial intelligence algorithm is trained to match preprocessed fourth Doppler ultrasound measurement results with labeled vessel locations based on cerebrovascular anatomical data.

12. The method of claim 7, wherein the information on microemboli within the subject comprises the presence of microemboli, a detection location of microemboli, a detection frequency of microemboli, an MES signal intensity, or a combination thereof.

13. A non-transitory computer-readable recording medium storing a computer program for executing the method according to claim 1.
